(19) United States
(12) Patent Application Publication
(10) Pub. No.: US 2003/0093111 A1
(43) Pub. Date: May 15, 2003

(54) Device for vaso-occlusion and interventional therapy

(75) Inventors: Christopher G.M. Ken, San Mateo, CA (US); Tina J. Patel, San Carlos, CA (US)

Correspondence Address: BANNER & WITCOFF, 1001 G STREET N W, SUITE 1100, WASHINGTON, DC, 20001, US

(73) Assignee: Concentric Medical, Mountain View, CA

(21) Appl. No.: 10/280,125

(22) Filed: Oct. 25, 2002

Related U.S. Application Data
(60) Provisional application No. 60/330,619, filed on Oct. 26, 2001.

(51) Int. Cl. A61M 29/00

(52) U.S. Cl. 606/200

(57) ABSTRACT

The invention provides a vaso-occlusive device having a braid comprising a bioabsorbable or bioactive material placed over and retaining a helical metallic coil. The braid or the coil may have one or more fibrous elements attached. A helical metallic coil comprising one or more attached fibrous elements is also claimed and described. Corresponding methods of making these devices and of treating patients having abnormal blood flow by implanting such devices at a site of abnormal blood flow are also claimed and described.

### CROSS-REFERENCE TO RELATED APPLICATION

This application claims benefit of U.S. Provisional Patent Application No. 60/330,619, filed on Oct. 26, 2001, by the same inventors and entitled “Device For Vaso-Occlusion And Interventional Therapy”. The full disclosure of this provisional application is incorporated hereby by reference.

The present invention relates to medical devices and methods for vaso-occlusion and interventional therapy.

### BACKGROUND OF THE INVENTION

Ruptured blood vessels in the brain cause an acute condition known as hemorrhagic stroke. Ruptures or strokes can occur with a number of vascular abnormalities including arterio venous malformation (AVM), aneurysm (a ballooning of the arterial wall), fistula, or a burst blood vessel. In addition, abnormal vasculature is generated in the process of tumor growth and tumors including brain tumors are highly vascularized entities requiring larger than normal blood flow to sustain the tumor.

Over 400,000 persons worldwide, and 125,000 persons in the U.S. annually experience some form of hemorrhagic stroke or blood vessel rupture in the brain. It would clearly be a benefit to the medical community and the field of interventional neurology to continue expanding and developing devices and/or agents for use in interventional neurology treatments for strokes and tumors. Interventional therapy can be applied to tumors in most locations in the body and is not limited to brain tumors. Interventional therapy seeks to reduce the blood flow and thus interrupt tumor growth upon the implantation of a vaso-occlusive device.

Endovascular therapy for vaso-occlusion has included injectable agents, balloon-type occlusive devices, and mechanical vaso-occlusive devices such as metal coils. A description of these agents and devices is included in the background section of U.S. Pat. No. 4,994,069. Currently, coils for aneurysms and polyvinyl alcohol (PVA) particles for AVMs are FDA approved preventative therapies. Cyanoacrylate glue for AVMs is also proposed and pending approval. Cyanoacrylate has disadvantages that include a propensity for the material to break away and enter the blood stream and affect healthy tissue.

Many vaso-occlusive devices contain biodegradable or other materials to increase thrombogenicity such as coating materials over the coil, e.g. metal particles, silicone, rubber or polymers. The coatings or additions may provide additional thrombogenicity to the device. The disadvantage of a coated device is that the device is generally one unit, a coated article, and the thrombogenicity maybe provided by the additional elements using the coil as a shape. However, a coated article does not necessarily provide blood flow access to the naked coil, thus eliminating any cumulative effects that might be gotten if the blood could access both the naked coil and the coating. With a coated device, the blood flow accesses only the coating.

Thus, it would be desirable to develop a vaso-occlusive device for treating abnormal blood flow by designing a device that provides maximal use of the different elements and configurations available for such devices, thus increasing the thrombogenicity achievable using the device.

### SUMMARY OF THE INVENTION

The invention provides a vaso-occlusive device comprising a helical coil wound from a filament of metallic wire; a flexible braid comprising a bioabsorbable material, the braid having a lumen for containing the helical coil, wherein the braid is positioned over the helical coil like a sleeve, and at least a first end of the braid is closed to form a closure to retain the helical coil inside the lumen of the braid.

The invention further provides a method of making a vaso-occlusive device comprising: providing a helical coil wound from a filament of metallic wire, sliding a tubular braid comprising a bioabsorbable material over the helical coil, and closing at least a first end of the braid to form a closure to retain the helical coil inside the lumen of the braid.

The invention further provides a vaso-occlusive device comprising a helical coil wound from a filament of metal; and one or more fibrous elements comprising a bioabsorbable or bioactive material, attached to or extending from the coil.

The invention further comprises a method of making a vaso-occlusive device comprising providing a helical coil wound from a filament of metallic wire, sliding a tubular braid comprising a bioabsorbable or bioactive material over the helical coil, and closing at least a first end of the braid to form a closure to retain the helical coil inside the lumen of the braid.

The invention also provides a method of treating a patient having abnormal blood flow at a site comprising: providing a helical coil wound from a filament of metallic wire and a flexible braid comprising a bioabsorbable material, the braid having a lumen for containing the helical coil, wherein the braid is positioned over the helical coil like a sleeve, and at least a first end of the braid is closed to form a closure to retain the helical coil inside the lumen of the braid; and implanting said coil at the site of abnormal blood flow in the patient.

A method of treating a patient having abnormal blood flow at a site comprises providing a helical coil wound from a filament of metallic wire and a flexible braid comprising a bioabsorbable or bioactive material, the braid having a lumen for containing the helical coil, wherein the braid is positioned over the helical coil like a sleeve, at least a first end of the braid is closed to form a closure to retain the helical coil inside the lumen of the braid, and attached to the braid or the coil is one or more fibrous elements comprising a bioabsorbable or bioactive material; and implanting said coil at the site of abnormal blood flow in the patient.

A method of treating a patient having abnormal blood flow at a site comprising: providing a helical coil wound from a filament of metallic wire comprising one or more fibrous elements comprising a bioabsorbable or bioactive material attached to the coil; and implanting said coil at the site of abnormal blood flow in the patient.

### BRIEF DESCRIPTION OF THE DRAWINGS

FIG. 1A shows a helical coil wound from a filament of metallic wire. FIG. 1B shows a multifilament metallic strand wound in a helical coil formation. FIG. 1C shows a cross-section of the coil shown in FIG. 1B.

FIG. 2A shows a flexible braid having a lumen. FIG. 2B shows the flexible braid having a closure at one end. FIG. 2C shows the braid of FIG. 2B having fibers forming a sleeve.

FIG. 3A shows a straightened or primary shape of a helically wound coil. FIG. 3B shows a secondary or relaxed shape of a “deployed” shape.

FIG. 4A shows a synched braid over a helical coil with one end synched and the second end attached (removably) to the inside walls of the deployment device. FIG. 4B shows a braid synched at both ends containing in its lumen a helical coil.

FIG. 5A shows plug at both ends of the device outside the helical lumen of the coil or the lumen of the braid. The braid is attached to the plugs at both ends of the device.

FIG. 5B shows a plug inside a helical lumen of the coil and the lumen of the braid. The plug and braid are attached by heat, adherence or mechanical fixation.

FIG. 6A shows a helical coil wound from a filament of metallic wire having several fibrous elements attached to the coil at intervals. FIG. 6B shows a multifilament metallic strand wound in a helical coil formation with another configuration of attached fibrous elements at intervals.

FIG. 7 shows a synched braid over a helical coil having fibrous elements attached to the braid at intervals.

### DETAILED DESCRIPTION

The following embodiments and examples are offered by way of illustration and not by way of limitation.

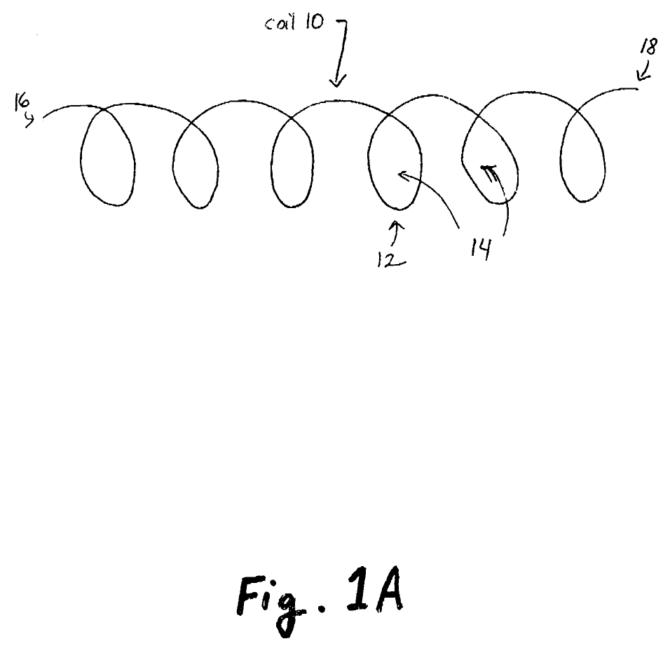

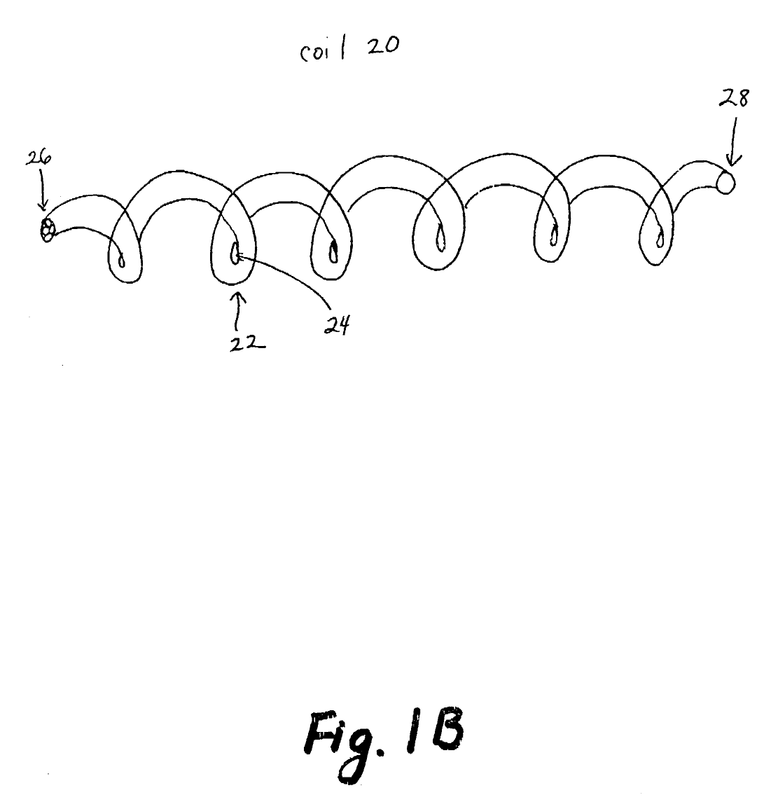

Turning first to the figures, FIG. 1A depicts a typical coil for use as an interior coil. The coil of FIG. 1A is a metallic coil 10 having a helical turn 12. The coil has lumen 14, created after multiple helical turns like turn 12. The coil is depicted in a straightened or pre-deployed state and as such as a first end 16 and a second end 18. The dimensions of the coil include an outside diameter in the range from about 0.003 inches to about 0.050 inches, or dimensions sufficient to include the coil within a delivery device for deploying the article into the patient. The length of the coil will typically be in a range from about 1 mm to about 5 meters. Frequently the length of the coil will depend on such variables as the capacity of the delivery device, the actual or estimated size of the target site for delivery in the patient, the extent of the bleeding, and other factors. The metallic wire can comprise a metal selected from the group consisting of platinum, stainless steel, nickel-titanium alloy, tungsten, gold, rhenium, palladium, rhodium, ruthenium, titanium, nickel, and alloys thereof.

FIG. 1B depicts a coil 20 having a multifilament metallic strand which forms the coil, forming the interior coil with helical turns. The coil 20 has helical turn 22, and lumen 24, a first end 26, and a second end 28. FIG. 1C depicts a cross-section of coil 20 and further identifies metallic filaments in a bundle 30, having such metallic strands as filament 32. The filaments are radio-opaque metallic filaments in a bundle that form a strand 34. The strand 34 can then form the coil 20 with helical turns such as turn 22 and a lumen such as lumen 24.

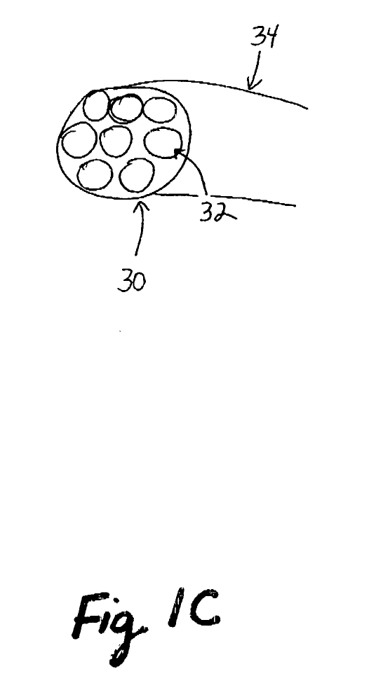

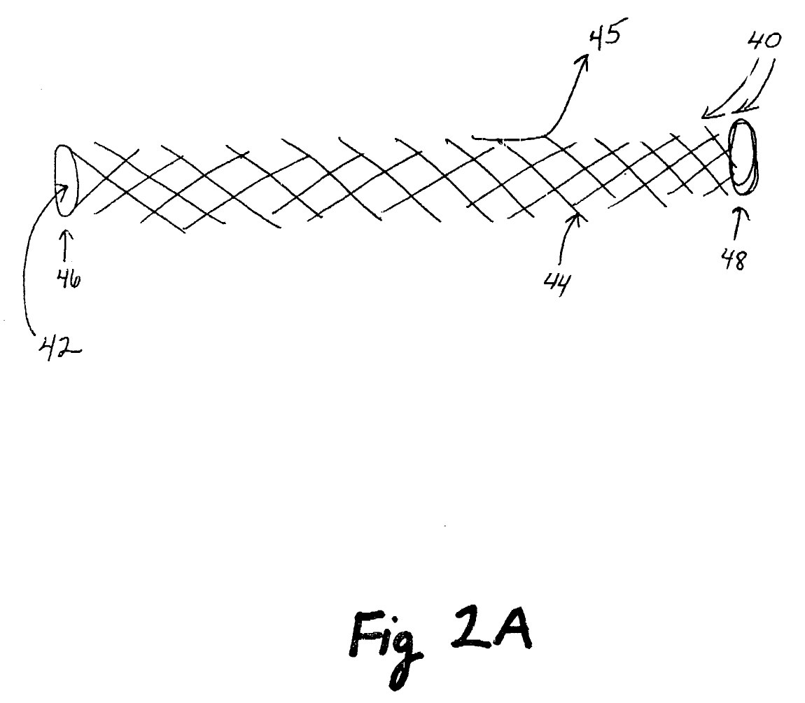

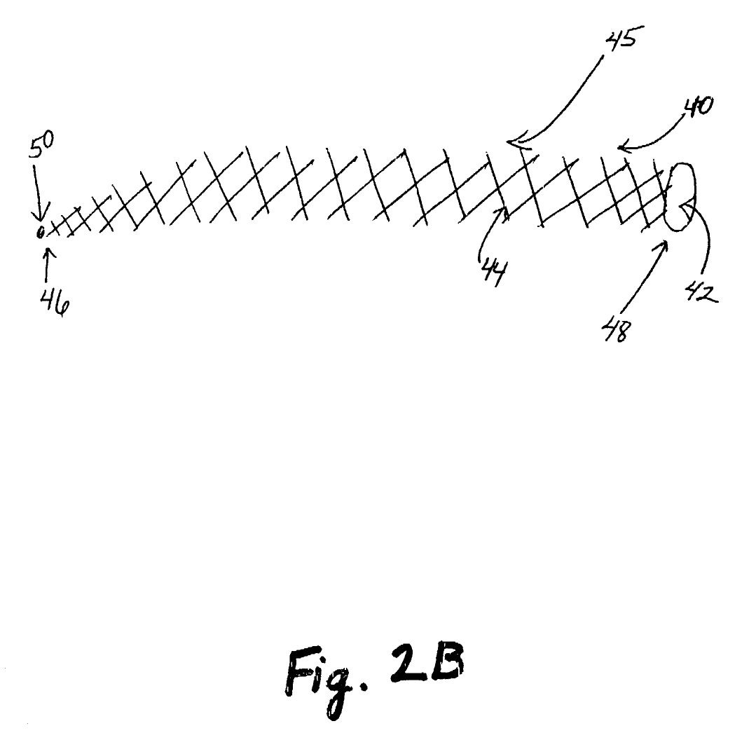

FIG. 2A depicts woven braid 40 which forms a sleeve 45 having a lumen 42. The braid is composed of multiple fibers like woven fiber 44. The braided sleeve 45 has a first end 46 and a second end 48. Both ends as depicted in this figure are open and the sleeve does not contain a coil. The fibers such as fiber 44 are made of a bioabsorbable material. Different fibers in the braid 40 can be made from different bioabsorbable materials. The bioabsorbable material can be a polymer.

FIG. 2B depicts braid 40 having fibers such as fiber 44 forming sleeve 45 having lumen 42 with first end 46 at which a closure 50 of the braided sleeve is located. End 48 is not closed. FIG. 2C depicts braid 40 having fibers such as fiber 44 forming sleeve 45 having lumen 42 with first end 46 at which a closure 50 of the braided sleeve is located. Closure 52 is located at end 48. Coil 10 is placed in the lumen 42 of sleeve 45, the coil itself comprising a lumen 14 and having helical turns such as turn 12 while in the sleeve in the stretched state prior to deployment.

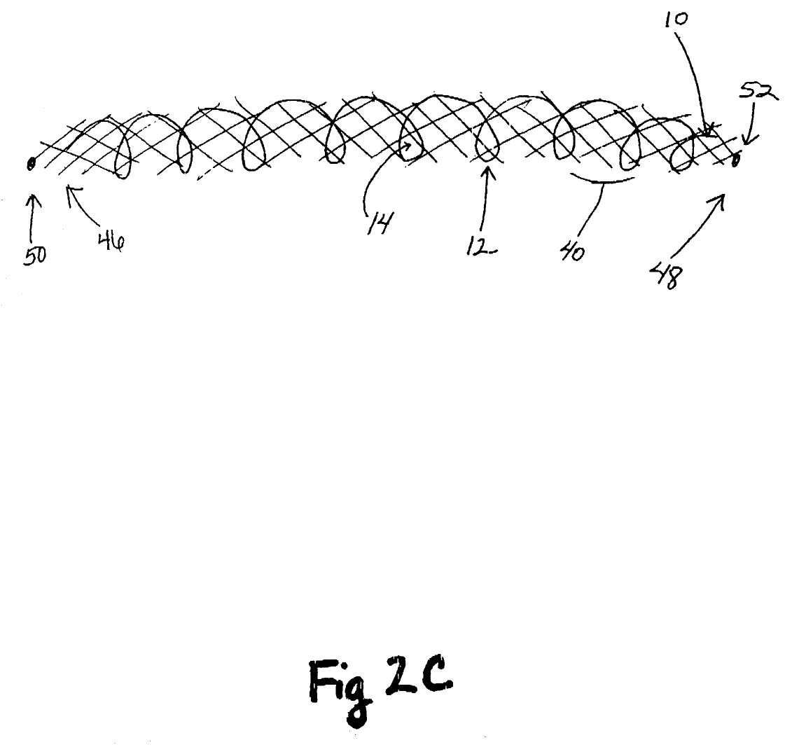

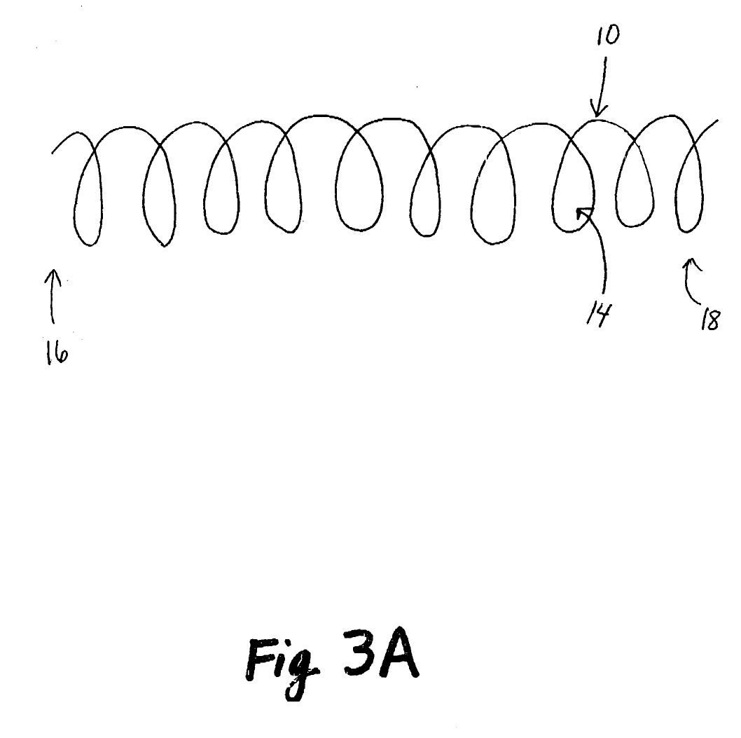

FIG. 3 depicts the pre-deployed or straightened state of a helical coil (FIG. 3A) and the post-deployed or relaxed state of the helical coil (FIG. 3B) such that the coil 10 forms a shape to occupy a target site of abnormal bleeding in the patient. Pre-deployed coil 10 (FIG. 3A) comprises a lumen 14 and has an end 16 and a second end 18. Post-deployed coil 10 (FIG. 3B) comprises a secondary shape having an end 16 and a second end 18, and multiple coilings upon coilings in between.

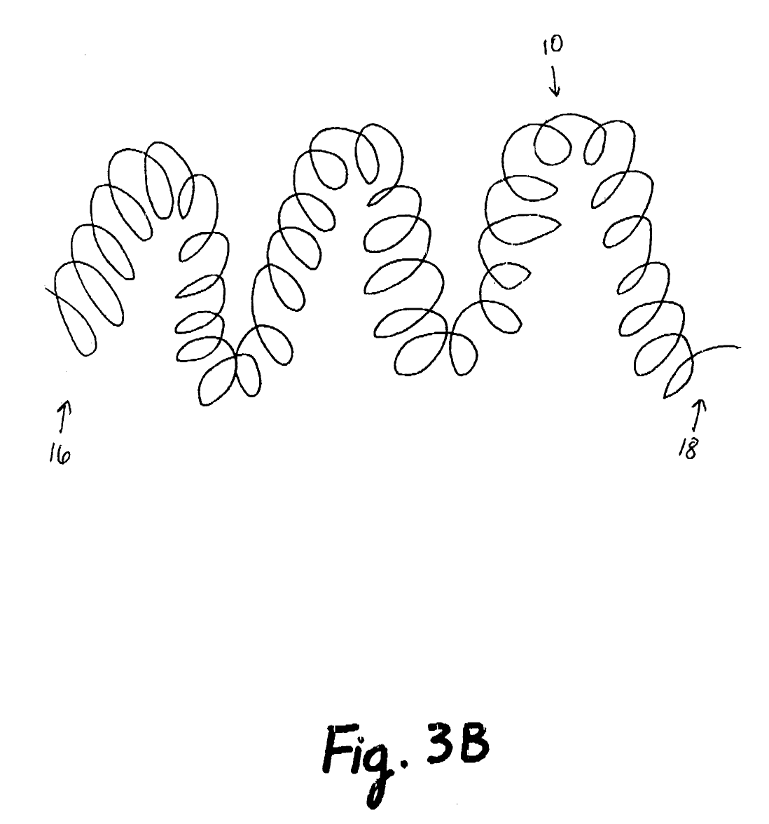

FIG. 4A depicts coil 10 surrounded by sleeve 45 comprising a woven braid 40 of filaments 44 and having a lumen 42. Sleeve 45 has a closure 50 at end 46. End 48 is open and portions of the sleeve at end 48 are attached to the deploy device wall 62 at attachment point 60. FIG. 4B depicts a sleeve 45 over a coil 10 having two closed ends, 50 and 52. Braid 40 made of filaments 44 surround and contain coil 10.

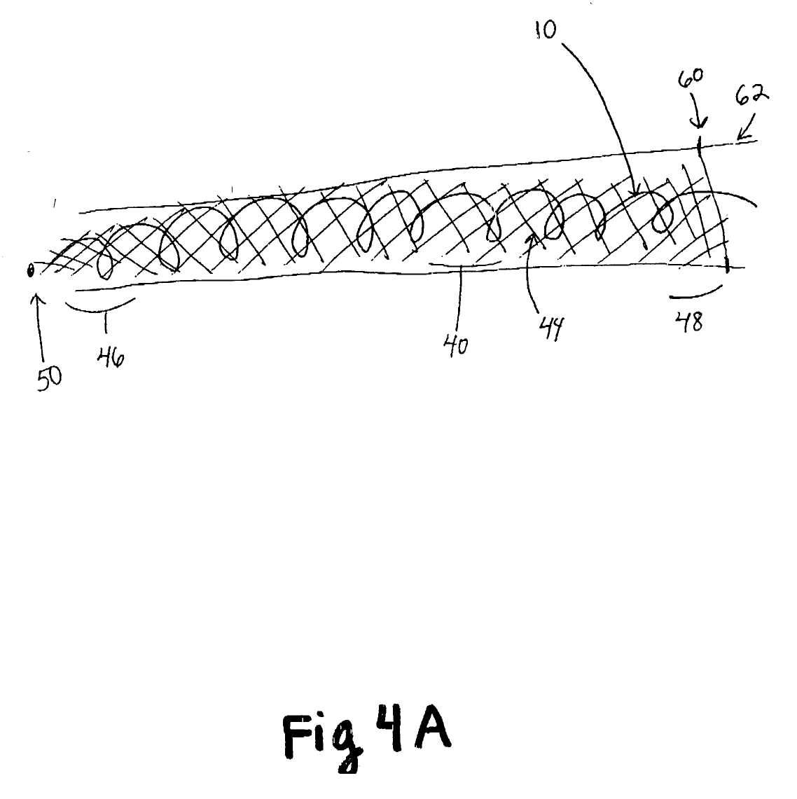

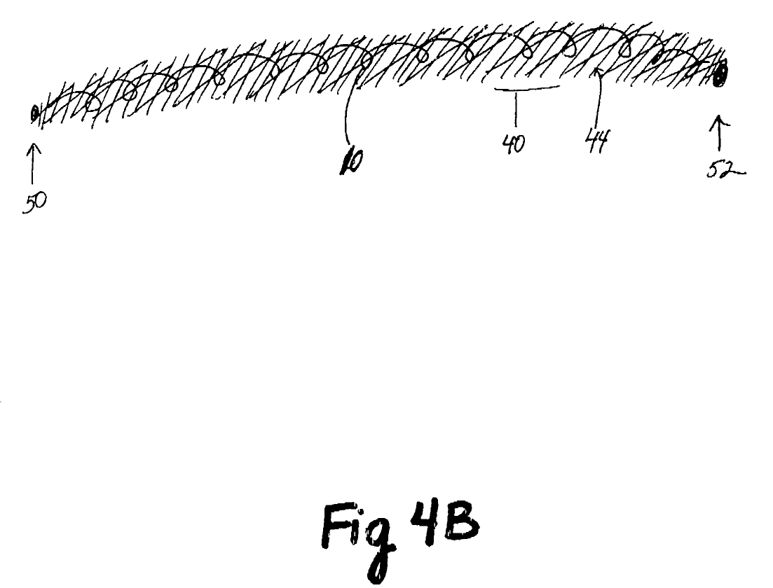

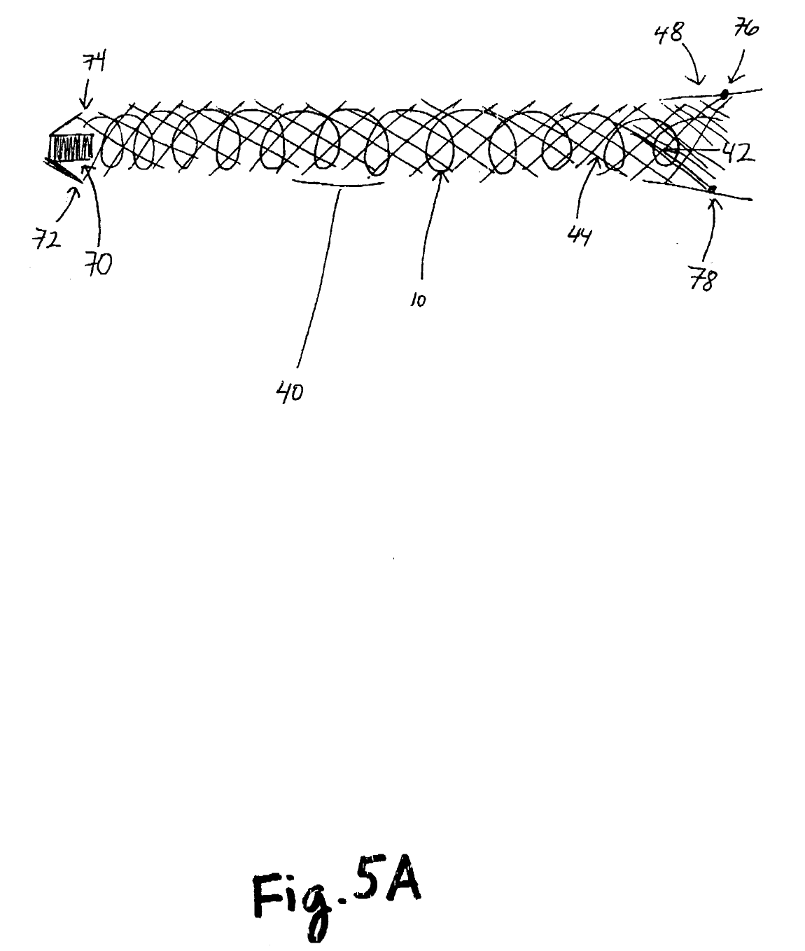

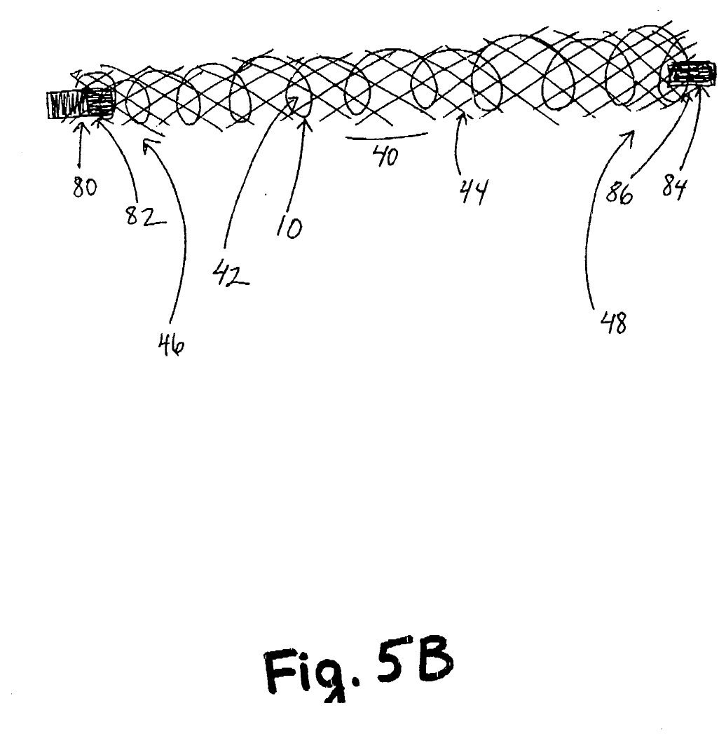

FIG. 5A depicts a sleeve 45 over coil 10 having a plug-like attachment at end 46. Plug 70 sits at the end of end 46 and is attached to the braided sleeve 45 at attachment 72 on the plug. Plug 70 rests outside lumen 42 of the sleeve 45 and outside of lumen 14 of the coil 10. End 48 comprising an open configuration where portions of the sleeve 45 are attached to the deployment device at temporary attachment points 76 and 78. Lumen 42 is therefore open prior to delivery.

FIG. 5B depicts sleeve 45 over coil 10, wherein the sleeve 45 has lumen 42 in which coil 10 having lumen 14 rests. Braid 40 attaches to plug 80 at attachment 82 at end 46. Plug 80 rests inside lumen 42 and lumen 14 without being affixed to coil 10. Plug 84 at end 48 similarly attaches to braid 40 at attachment 86 and rests in the lumen 42 of the sleeve 45 and the lumen 14 of the coil 10. Plug 84 is attached to braid 40 but is not attached to coil 10.

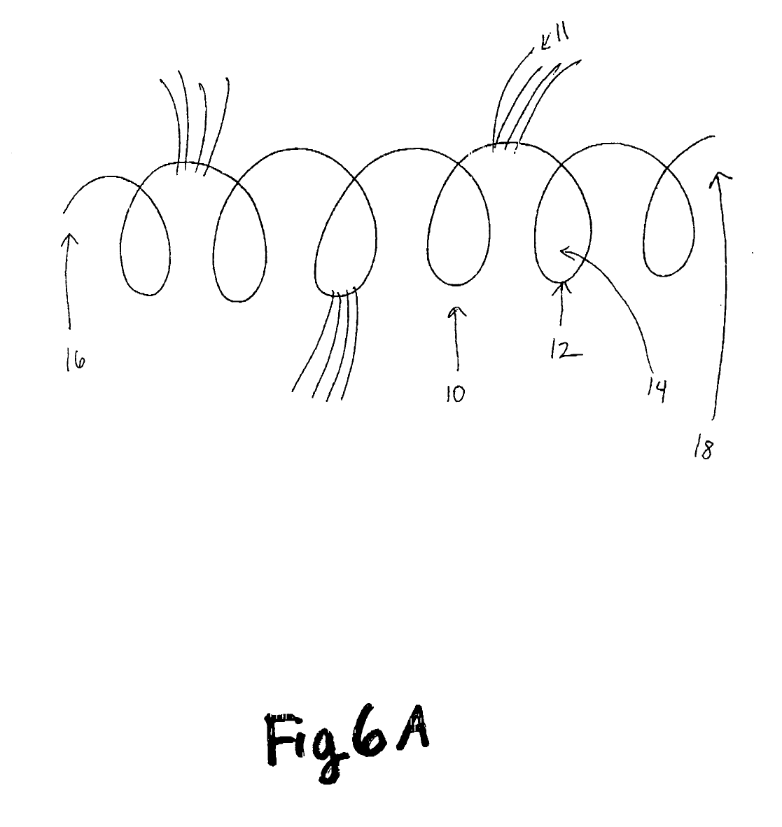

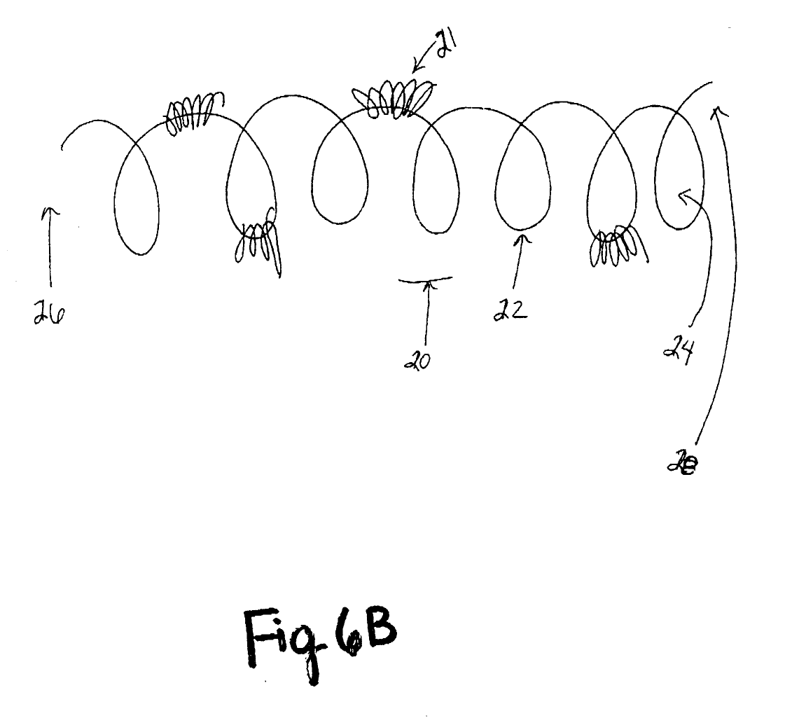

FIG. 6A shows a helical coil wound from a filament of metallic wire having several fibrous elements attached to the coil at intervals. FIG. 6B shows a multifilament metallic strand wound in a helical coil formation with another configuration of attached fibrous elements at intervals. FIG. 6A depicts a typical coil for use as an interior coil. The coil of FIG. 6A is a metallic coil 10 having a helical turn 12. The coil has lumen 14, created after multiple helical turns like turn 12. The coil is depicted in a straightened or pre-deployed state and as such as a first end 16 and a second end 18. The dimensions of the coil include an outside diameter in the range from about 0.003 inches to about 0.050 inches, or dimensions sufficient to include the coil within a delivery device for deploying the article into the patient. The length of the coil will typically be in a range from about 1 mm to about 5 meters. Frequently the length of the coil will depend on such variables as the capacity of the delivery device, the actual or estimated size of the target site for delivery in the patient, the extent of the bleeding, and other factors. In addition, the coil 10 has fibrous elements 11 attached at various intervals of the helical turns 12.

FIG. 6B depicts a coil having a multifilament metallic strand which forms the coil, forming the interior coil with helical turns. The coil 20 has helical turn 22, and lumen 24, a first end 26, and a second end 28. Fibrous elements 21 are attached at helical turns such as 22 at intervals along coil 20. The filaments of the metallic strand in a multifilament strand can comprise metal selected from the group consisting of platinum, stainless steel, nickel-titanium alloy, tungsten, gold, rhenium, palladium, rhodium, ruthenium, titanium, nickel, and alloys thereof.

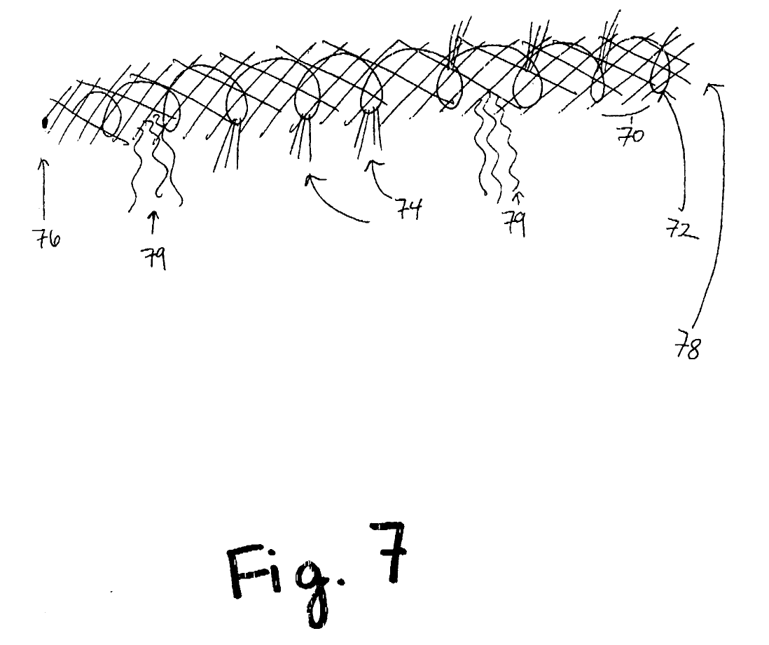

FIG. 7 shows a synched braid 70 over a helical coil 72 having fibrous elements 74 attached to the braid 70 at intervals. The device has ends 76 and 78. Alternative types of fibrous elements 79 are also shown attached to the braid 70.

A vaso-occlusive device can comprise a helical coil wound from a filament of metallic wire. The metal of the filament of metallic wire can comprise a metal selected from the group consisting of platinum, stainless steel, nickel-titanium alloy, tungsten, gold, rhenium, palladium, rhodium, ruthenium, titanium, nickel, and alloys thereof. Over the helical coil of metallic wire can be slid a previously assembled flexible braid comprising a bioabsorbable or bioactive material. The braid can comprise a lumen for containing the helical coil. The braid can be positioned over the helical coil like a sleeve. The braid might also be tied closed along the length of the braid thereby covering the central coil. One end of the braid can be closed to form a closure to retain the helical coil inside the lumen of the braid. The helical coil can be heat treated to form a secondary shape. For example, a primary, stretched, pre-deployment shape can be a helical coil. The stretched shape would be assumed in the delivery device and the heat-treated coil would relax into the secondary shape after deployment or after leaving the delivery device. A secondary, relaxed or deployed shape can be for example a cloverleaf or further coiled coil that would form in the coil after deployment into the target site of the patient. The advantage of heat-treating the helical coil or primary coil is to introduce a controlled tension in the shape which is released after the device is deployed and provides an additional complexity to the configuration of the device which complexity aids in promoting thrombogenicity.

The other end or second end of the braid can be held in a delivery device to form a temporary closure to retain the helical coil inside the lumen of the braid prior to delivery of the device. The second end of the braid can be closed to form a closure to retain the helical coil inside the lumen of the braid. Either or both closures of the braid can be provided by any means possible to close the braid and contain the coil within the sleeve. The coil is thus contained within the sleeve by friction, and in contact with the braid of the sleeve by friction, but the coil is not permanently attached to the sleeve.

Closure of the braid to encase or house the coil can be provided by a synched portion at one or both ends of the braid. Synching can be accomplished by pulling the woven fibers of the braid at the end together to close the braided sleeve down. The synched ends can then be tied (mechanical fixation), melted (heat closure), or adhered together (e.g. with a glue or other adherence material) to make a permanent closure of the end. Other fixation means may also apply to the synched closure if appropriate. Thus, the synched portion can be heated to seal the synched portion or portions. The heating can melt the bioabsorbable material to a closure. The synched portion can be mechanically fixed with a tie or other mechanical fixation at one or both synched ends. Mechanical fixation can included pulling a woven fiber of the braid or tying-off the end with a fiber or wire, for example. At least one end of the braid can be attached to a plug to provide closure of one or both of the sleeve's ends. The plug can comprise a bioabsorbable or bioactive monofilament. The sleeve end or braid can be attached to the plug to provide closure for the end of the braided sleeve. Each end of the braided sleeve can have a plug. An end of the braid can be attached to the plug by heat to melt the braided sleeve's ends onto the plug to form the closure. Adhesion or mechanical fixation to the plug can also provide contact of the braid with the plug and form a closure of the braided sleeve.

The plug may rest at the end of the braid, but inside the lumen of the braid and inside the lumen of the coil. The braid or sleeve can be attached to the plug, and not attached to the coil, but the plug may be tucked inside the lumen of the device. The coil is not attached to the plug. The braid can be affixed to an end of the braid by either heat, adhesive or mechanical means as describe before.

The vasoocclusive device comprising a braided sleeve over a helical coil, can further comprise one or more fibrous elements extending from the braid. The fibrous elements can be made of a bioabsorbable or bioactive material. In addition, or alternatively, bioabsorbable or bioactive fibrous elements can attach to the helical coil resting inside the braid, and the fibrous elements can extend out beyond the braid. Such a configuration of bioabsorbable or bioactive material may further enhance thrombogenicity of the device and in general the bioactive nature of the device once implanted at a site in the body.

The invention also includes a vaso-occlusive device comprising a helical coil wound from a filament of metal and one or more fibrous elements comprising a bioabsorbable or bioactive material, attached to or extending from the coil. The metal of the helical coil can comprise a metal selected from the group consisting of platinum, stainless steel, nickel-titanium alloy, tungsten, gold, rhenium, palladium, rhodium, ruthenium, titanium, nickel, and alloys thereof.

In any of the devices or embodiments described, the helical coil can comprise a multifilament metallic strand. Such a coil is radio-opaque and can be followed during delivery and thereafter inside the body of the patient. The filaments of the coil can comprise such materials as are generally used in embolic coils, for example, platinum, stainless steel, nickel, titanium, tungsten, gold, rhenium, palladium, rhodium, ruthenium, and alloys thereof, e.g. nickel-titanium alloys and others.

The bioabsorbable or bioactive material that comprises the braid of the sleeve that passes over the coil or the fibrous elements can comprise any bioabsorbable or bioactive material that can be received and absorbed in the body without adverse affects. The bioabsorbable material can comprise a bioabsorbable polymer. The bioactive material can comprise a polymer that bioabsorbs and generates a bioactive response at a site of implantation in the process. The bioactive material can comprise a polymer comprising a bioactive agent that generates scar tissue in the healing process. The bioabsorbable or bioactive material can comprise one or more agents that bioabsorbs or is otherwise bioactive at the site of implantation.

Preferably the bioabsorbtion of the material of the braid or fibrous elements will aid or facilitate thrombogenic activity at the site of delivery or deposit of the device in the patient. The bioabsorbable or bioactive material can comprise a bioabsorbable polymer. The bioabsorbable polymer can be, for example, a polymer selected from the list as follows, formed into a fiber and woven into a braid to form the sleeve that slides over the coil: polyacrylamide (PAAM), poly (N-isopropylacrylamine) (PNIPAM), poly (vinylmethylether), poly (ethylene oxide), poly (vinylalcohol), poly (ethyl (hydroxyethyl) cellulose), poly(2-ethyl oxazoline), Polylactide (PLA), Polyglycolide (PGA), Poly(lactide-co-glycolide) PLGA, Poly(e-caprolactone), Polydiaoxanone, Polyanhydride, Trimethylene carbonate, Poly(β-hydroxybutyrate), Poly(g-ethyl glutamate), Poly(DTH-iminocarbonate), Poly(bisphenol A iminocarbonate), Poly(orthoester) (POE), Polycyanoacrylate (PCA), Polyphosphazene, Polyethyleneoxide (PEO), Polyethylglycol (PEG), Polyacrylacid (PAA), Polyacrylonitrile (PAN), Polyvinylacrylate (PVA), Polyvinylpyrrolidone (PVP), a copolymer, or a blend of two or more polymers. These polymers can be blended and formed so that they form a semi-rigid, but flexible fibers that can be woven into a braid and the braid can be formed into a slidable sleeve.

The bioabsorbable or bioactive material of the braid or fibrous elements can also be, for example, a natural polymer. The natural polymer can be selected from the group consisting of collagen, silk, fibrin, gelatin, hyaluron, cellulose, chitin, dextran, casein, albumin, ovalbumin, heparin sulfate, starch, agar, heparin, alginate, fibronectin, fibrin, pectin, elastin, keratin, a copolymer, or a blend of polymers. The braid or fibrous elements can thus also be, for example, a combination of fibers of different materials, all either bioabsorbable or bioactive or both, some bioabsorbable polymers, some comprising bioactive agents that act in addition to the bioabsorbtion effects.

Additionally, U.S. Pat. No. 6,184,348 describes production of novel polymers using recombinant techniques, and also integration of bioactive agents potentially useful at a site of implantation in the patient. U.S. Pat. No. 6,184,348 also describes spinning applicable here as a way to incorporate a bioactive agent. A bioactive agent can be incorporated into the bioabsorbable material to increase thrombogenicity, or perform other biologically relevant and helpful functions at the site of the delivery of the device.

The bioactive agent that can be added to the braid or fibrous elements to supplement the activity of bioabsorbtion and the bioactivity ensuing from the bioabsorbtion can be an agent that promotes any biological activity desired at the site of abnormal blood flow. Some possible desired biological activities can include (but are not limited to) for example, occluding blood flow, adhering the device at the site of implantation, building a damaged vascular wall, regressing capillary dilation, inhibiting capillary dilation, regressing an AVM, inhibiting an AVM, regressing tumor growth, or inhibiting tumor growth, to name a few but not all of the possible or desired biological activities that could be present in any given selected bioactive agent.

The bioactive agent can, accordingly, be selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

The amount of bioactive agent used will preferably be an amount sufficient for the agent to be effective at the site of implantation for the biological activity expected from the agent. What would be an effective amount for any given agent or agents can be determined on an agent by agent basis, taking into account standard, known parameters of any given bioactive agents such as potency, available concentration, and volume of space within the patient to be targeted for the desired effect. Efficacy and proper dosage can be determined by routine assay specific for the bioactive agent selected using for example standard known assays provided in well known frequently used laboratory assay and protocol manuals for identifying activity and quantifying potency of molecules and cells. The vaso-occlusive device can also comprise a radio pacifier.

The invention also provides a method of making a vaso-occlusive device comprising providing a helical coil wound from a filament of metallic wire, sliding a tubular braid comprising a bioabsorbable or bioactive material over the helical coil, and closing at least a first end of the braid to form a closure to retain the helical coil inside the lumen of the braid. The metal of the metallic wire can comprise a metal selected from the group consisting of platinum, stainless steel, nickel-titanium alloy, tungsten, gold, rhenium, palladium, rhodium, ruthenium, titanium, nickel, and alloys thereof. The bioabsorbable or bioactive material can comprise a bioabsorbable polymer, for example such as those listed herein. The helical coil can be heat treated to form a secondary shape as described above. The secondary shape formed by heat treatment is assumable after sliding the braid over the coil, and after delivery of the device to the patient.

Closing can comprise a procedure including one or more of the following: synching at least one end of the braid to form a closure of the braid, providing a monofilament plug comprising a bioabsorbable material affixed to at least one end of the braid to form a closure of the braid, or placing a plug comprising a bioabsorbable material into a lumen of the tubular braid and a lumen of the helical coil wherein the plug is affixed to an end of the braid by heat, adherence, or mechanical fixation. In the method of making the device, providing a helical coil can comprises winding a primary coil made from a multifilament strand.

As described above, the bioabsorbable or bioactive material can comprise a bioabsorbable polymer. The bioactive material can comprise a polymer having a bioactive agent that generates scar tissue in the healing process. The bioactive material can comprise a polymer that bioabsorbs and generates a bioactive response at a site of implantation in the process.

The method of making a vaso-occlusive device can further comprise attaching one or more fibrous elements comprising a bioabsorbable or bioactive material to the braid. The method can also comprise attaching one or more fibrous elements comprising a bioabsorbable or bioactive material to the coil. In the latter case, the fibrous elements can extend from the coil through the braid to the external portion of the device.

A method of making a vaso-occlusive device can comprise providing a helical coil wound from a filament of metal wire, and attaching one or more fibrous elements to the coil, wherein the fibrous element comprises a bioabsorbable or bioactive material. The metal for the coil can be selected from the group consisting of platinum, stainless steel, nickel-titanium alloy, tungsten, gold, rhenium, palladium, rhodium, ruthenium, titanium, nickel, and alloys thereof. The helical coil can comprise a multifilament metal strand.

In addition, the invention also provides a method of treating a patient having abnormal blood flow at a site comprising implanting said coil at the site of abnormal blood flow in the patient a device comprising a helical coil wound from a filament of metallic wire and a flexible braid comprising a bioabsorbable or bioactive material, with the braid having a lumen for containing the helical coil, wherein the braid is positioned over the helical coil like a sleeve, and one or both ends of the braid is closed to form a closure to retain the helical coil inside the lumen of the braid. The site of abnormal blood flow, and thus a potential site for delivery of the device into a patient can comprise a condition selected from the group consisting of ruptured blood vessels, aneurysms, arterio venus malformations (AVMs), fistulas, benign tumors, and malignant tumors.

Another method of treating a patient having abnormal blood flow at a site can comprise implanting at a site of abnormal blood flow a helical coil wound from a filament of metallic wire and a flexible braid comprising a bioabsorbable or bioactive material, the braid placed over and containing the helical coil, like a sleeve, where attached to the braid or the coil is one or more fibrous elements comprising a bioabsorbable or bioactive material. Alternatively, the method of treatment can comprise implanting a device comprising a helical coil wound from a filament of metallic wire comprising one or more fibrous elements comprising a bioabsorbable or bioactive material attached to the coil.

The site of abnormal blood flow in any case of treatment method can comprise (but is not limited to) a condition selected from the group consisting of ruptured blood vessels, aneurysms, arterio venus malformations (AVMs), fistulas, benign tumors, and malignant tumors.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A vaso-occlusive device comprising:
a helical coil wound from a filament of metallic wire;
a flexible braid comprising a bioabsorbable or bioactive material, the braid having a lumen for containing the helical coil, wherein the braid is positioned over the helical coil like a sleeve, and at least a first end of the braid is closed to form a closure to retain the helical coil inside the lumen of the braid.

2. A vaso-occlusive device as in claim 1, wherein the helical coil is heat treated to form a secondary shape.

3. A vaso-occlusive device as in claim 1, wherein a second end of the braid is held in a delivery device to form a temporary closure to retain the helical coil inside the lumen of the braid prior to delivery of the device.

4. A vaso-occlusive device as in claim 1, further comprising that a second end of the braid is closed to form a closure to retain the helical coil inside the lumen of the braid.

5. A vaso-occlusive device as in claim 1, wherein the coil is in contact with the braid by friction.

6. A vaso-occlusive device as in claim 3, wherein the coil is in contact with the braid by friction.

7. A vaso-occlusive device as in claim 1, wherein the closure is provided by a synched portion at an end of the braid.

8. A vaso-occlusive device as in claim 7, wherein the closure is provided by a synched portion at each end of the braid.

9. A vaso-occlusive device as in claim 7, wherein the synched portion is heated to seal the synched portion.

10. A vaso-occlusive device as in claim 8, wherein the synched portions are heated to seal the synched portions.

11. A vaso-occlusive device as in claim 7, wherein the synched portion is mechanically fixed with a tie or other mechanical fixation at the synched end.

12. A vaso-occlusive device as in claim 8, wherein the synched portions are mechanically fixed with a tie or other mechanical fixation at the synched ends.

13. A vaso-occlusive device as in claim 1, wherein at least one end of the braid is attached to a plug comprising a bioabsorbable or bioactive monofilament, wherein the attachment provides closure for an end of the braid.

14. A vaso-occlusive device as in claim 13, wherein an end of the braid is attached to the plug by heat.

15. A vaso-occlusive device as in claim 13, wherein an end of the braid is adhered to the plug to provide closure for the end of the braid.

16. A vaso-occlusive device as in claim 13, wherein an end of the braid is mechanically fixed to the plug to provide closure for the end of the braid.

17. A vaso-occlusive device as in claim 1, wherein the ends of the braid are synched to hold the coil in place and the coil contacts the braid by friction.

18. A vaso-occlusive device as in claim 13, wherein a plug at an end of the braid rests inside the lumen of the braid and a lumen of the coil and is affixed to an end of the braid by either heat, adhesive or mechanical means.

19. A vaso-occlusive device as in claim 1, wherein the helical coil comprises a multifilament metallic strand.

20. A vaso-occlusive device as in claim 1, wherein the metallic wire comprises a metal selected from the group consisting of platinum, stainless steel, nickel-titanium alloy, tungsten, gold, rhenium, palladium, rhodium, ruthenium, titanium, nickel, and alloys thereof.

21. A vaso-occlusive device as in claim 1, wherein the helical coil comprises a multifilament metallic strand.

22. A vaso-occlusive device as in claim 1, wherein the bioabsorbable material comprises a bioabsorbable polymer.

23. A vaso-occlusive device as in claim 1, wherein the bioactive material comprises a polymer that bioabsorbs and generates a bioactive response at a site of implantation in the process.

24. A vaso-occlusive device as in claim 1, wherein the bioactive material comprises a polymer comprising a bioactive agent that generates scar tissue in the healing process.

25. A vaso-occlusive device as in claim 1, wherein the bioabsorbable or bioactive material comprises one or more agents that bioabsorbs or is otherwise bioactive at the site.

26. A vaso-occlusive device as in claim 1, further comprising one or more fibrous elements attached to or extending from the braid.

27. A vaso-occlusive device as in claim 1, further comprising one or more fibrous elements attached to or extending from the helical coil.

28. A vaso-occlusive device comprising:
a helical coil wound from a filament of metallic wire; and
one or more fibrous elements comprising a bioabsorbable or bioactive material, attached to or extending from the coil.

29. A vaso-occlusive device of claim 28, wherein the metal comprises a metal selected from the group consisting of platinum, stainless steel, nickel-titanium alloy, tungsten, gold, rhenium, palladium, rhodium, ruthenium, titanium, nickel, and alloys thereof.

30. A vaso-occlusive device as in claim 28, wherein the helical coil comprises a multifilament metallic strand.

31. A vaso-occlusive device as in claim 28, wherein the bioabsorbable material comprises a bioabsorbable polymer.

32. A vaso-occlusive device as in claim 28, wherein the bioactive material comprises a polymer comprising a bioactive agent that generates scar tissue in the healing process.

33. A vaso-occlusive device as in claim 28, wherein the bioactive material comprises a polymer that bioabsorbs and generates a bioactive response at a site of implantation in the process.

34. A method of making a vaso-occlusive device comprising:
providing a helical coil wound from a filament of metallic wire,
sliding a tubular braid comprising a bioabsorbable or bioactive material over the helical coil, and
closing at least a first end of the braid to form a closure to retain the helical coil inside the lumen of the braid.

35. A method as in claim 34, wherein the filament of metallic wire comprise a metal selected from the group consisting of platinum, stainless steel, nickel-titanium alloy, tungsten, gold, rhenium, palladium, rhodium, ruthenium, titanium, nickel, and alloys thereof.

36. A method as in claim 34, wherein the bioabsorbable material comprises a bioabsorbable polymer.

37. A method as in claim 34, wherein the bioactive material comprises a polymer comprising a bioactive agent that generates scar tissue in the healing process.

38. A method as in claim 34, wherein the bioactive material comprises a polymer that bioabsorbs and generates a bioactive response at a site of implantation in the process.

39. A method as in claim 34, further comprising heat treating the helical coil to form a secondary shape.

40. A method as in claim 39, wherein the secondary shape formed by heat treating is assumable after sliding the braid over the coil, and after delivery of the device to the patient.

41. A method as in claim 34, wherein closing comprises a procedure selected from the group consisting of synching at least one end of the braid to form a closure of the braid, providing a monofilament plug comprising a bioabsorbable or bioactive material affixed to at least one end of the braid to form a closure of the braid, and placing a plug comprising a bioabsorbable or bioactive material into a lumen of the tubular braid and a lumen of the helical coil wherein the plug is affixed to an end of the braid by heat, adherence, or mechanical fixation.

42. A method as in claim 34, wherein providing a helical coil comprises winding a primary coil made from a multifilament strand.

43. A method as in claim 34, further comprising attaching one or more fibrous elements comprising a bioabsorbable or bioactive material to the braid.

44. A method as in claim 34, further comprising attaching one or more fibrous elements comprising a bioabsorbable or bioactive material to the coil.

45. A method of making a vaso-occlusive device comprising:
providing a helical coil wound from a filament of metallic wire, and
attaching one or more fibrous elements to the coil, wherein the fibrous element comprises a bioabsorbable or bioactive material.

46. A method of claim 45, wherein the metal is selected from the group consisting of platinum, stainless steel, nickel-titanium alloy, tungsten, gold, rhenium, palladium, rhodium, ruthenium, titanium, nickel, and alloys thereof.

47. A method as in claim 45, wherein the coil comprises a multifilament metal strand.

48. A method of treating a patient having abnormal blood flow at a site comprising:
providing a helical coil wound from a filament of metallic wire and a flexible braid comprising a bioabsorbable or bioactive material, the braid having a lumen for containing the helical coil, wherein the braid is positioned over the helical coil like a sleeve, and at least a first end of the braid is closed to form a closure to retain the helical coil inside the lumen of the braid; and
implanting said coil at the site of abnormal blood flow in the patient.

49. A method as in claim 48, wherein the site of abnormal blood flow comprises a condition selected from the group consisting of ruptured blood vessels, aneurysms, arterio venus malformations (AVMs), fistulas, benign tumors, and malignant tumors.

50. A method of treating a patient having abnormal blood flow at a site comprising:
providing a helical coil wound from a filament of metallic wire and a flexible braid comprising a bioabsorbable or bioactive material, the braid having a lumen for containing the helical coil, wherein the braid is positioned over the helical coil like a sleeve, at least a first end of the braid is closed to form a closure to retain the helical coil inside the lumen of the braid, and attached to the braid or the coil is one or more fibrous elements comprising a bioabsorbable or bioactive material; and
implanting said coil at the site of abnormal blood flow in the patient.

51. A method of treating a patient having abnormal blood flow at a site comprising:
providing a helical coil wound from a filament of metallic wire comprising one or more fibrous elements comprising a bioabsorbable or bioactive material attached to the coil; and
implanting said coil at the site of abnormal blood flow in the patient.

52. A method as in claim 50, wherein the site of abnormal blood flow comprises a condition selected from the group consisting of ruptured blood vessels, aneurysms, arterio venus malformations (AVMs), fistulas, benign tumors, and malignant tumors.

53. A method as in claim 51, wherein the site of abnormal blood flow comprises a condition selected from the group consisting of ruptured blood vessels, aneurysms, arterio venus malformations (AVMs), fistulas, benign tumors, and malignant tumors.
